(19) United States
(12) Patent Application Publication
(10) Pub. No.: US 2023/0181552 A1
(43) Pub. Date: Jun. 15, 2023

(54) EXTERNAL PREPARATION OF IMIDACLOPRID, PREPARATION METHOD AND USE THEREOF

(71) Applicant: Qingdao Bolin Biological Technology Co., Ltd., Shandong (CN)

(72) Inventors: Yongda ZHAO, Shandong (CN); Ruiyuan Gao, Shandong (CN); Jiahui Wei, Shandong (CN); Xuan Ma, Shandong (CN); Xiaoyu Xu, Shandong (CN); Caichao Dong, Shandong (CN)

(73) Assignee: Qingdao Bolin Biological Technology Co., Ltd., Shandong (CN)

(21) Appl. No.: 17/665,366

(22) Filed: Feb. 4, 2022

(30) Foreign Application Priority Data
Dec. 13, 2021 (CN) 202111515465.7

(51) Int. Cl. A61K 31/4439; A61K 47/22; A61K 47/10; A61K 9/00; A61P 33/14

(52) U.S. Cl. CPC A61K 31/4439; A61K 47/22; A61K 47/10; A61K 9/0017; A61P 33/14

(57) ABSTRACT

Disclosed are an external preparation of imidacloprid, a preparation method and use thereof, which relates to the technical field of veterinary drugs. The present disclosure uses alcohol solvent as the dispersion medium of imidacloprid technical, which can ensure the stability of imidacloprid technical; The present disclosure uses one or more of dibutyl hydroxytoluene, hydroxymethyl, propyl hydroxybenzoate and butylated hydroxyanisole as the antioxidant, which can effectively prevent the oxidation of imidacloprid technical and prolong the preservation time of external preparations of imidacloprid; The present disclosure uses one or more of propylene carbonate, povidone and copovidone as the stabilizer, which can reduce the volatility of the solvent and maintain the stability of the dispersion system. At the same time, the adjuvants used in the present disclosure have the advantages of safety, no skin irritation, low cost, and are suitable for market promotion and application.

### CROSS REFERENCE TO RELATED APPLICATION(S)

This patent application claims the benefit and priority of Chinese Patent Application No. 202111515465. 7, filed on Dec. 13, 2021, the disclosure of which is incorporated by reference herein in its entirety as part of the present application.

### TECHNICAL FIELD

The present disclosure relates to the technical field of veterinary drugs, in particular to an external preparation of imidacloprid, a preparation method and use thereof.

### BACKGROUND ART

Lice, fleas, and ticks are widely distributed all over the world, whose hosts include rodents, humans, dogs, cats, horses, and cattle. Imidacloprid is a chlorinated nicotinic insecticide, which has a high affinity for postsynaptic nicotinic acetylcholine receptors in the central nervous system of insects, and can inhibit the activity of acetylcholine, leading to paralysis and death of parasites. Imidacloprid has insecticidal effects on adult fleas and juvenile fleas in the environment. Due to the weak affinity of imidacloprid to nicotinic receptor sites of mammals and the poor penetration of imidacloprid through the blood-brain barrier of mammals, it can be inferred that imidacloprid has almost no effect on the central nervous system of mammals. Safety studies of sublethal doses of imidacloprid for systemic use in rabbits, mice and rats have confirmed that imidacloprid has little pharmacological activity in mammals. Therefore, the application of imidacloprid on pets has a good application prospect.

Chinese patent application CN106727552A discloses a compound preparation of imidacloprid and permethrin, which can be applied to the treatment of pet parasitosis; Chinese patent application CN107157995A discloses a compound preparation of imidacloprid, doramectin, and pyrethroid compounds, which can be used to repel parasites inside and outside pets. However, the above imidacloprid preparations have the problem of poor stability, the shelf life is short, and they are easy to fail after being placed for a long time.

### SUMMARY

In view of this, the purpose of the present disclosure is to provide an external preparation of imidacloprid, a preparation method and use thereof. The external preparation of imidacloprid provided by the present disclosure has good stability.

In order to achieve the above purpose, the present disclosure provides the following technical schemes:

The present disclosure provides an external preparation of imidacloprid, including the following components in percentage by mass:

Imidacloprid technical 5-15%;

Antioxidant 0.05-0.2%;

Alcohol solvent 80-90%;

Stabilizer 4.95-10%;

The antioxidant is one or more of dibutyl hydroxytoluene, hydroxymethyl, propyl hydroxybenzoate and butylated hydroxy anisole;

The stabilizer is one or more of propylene carbonate, povidone and copovidone.

In some embodiments of the present disclosure, the external preparation of imidacloprid includes the following components in percentage by mass:

Imidacloprid technical 8-12%;

Antioxidant 0.1-0.15%;

Alcohol solvent 82-88%;

Stabilizer 6-8%.

In some embodiments of the present disclosure, the alcohol solvent is one or more of benzyl alcohol, propylene glycol and isopropanol.

In some embodiments of the present disclosure, the dosage form of the external preparation of imidacloprid is a drop.

The present disclosure provides a method for preparing the external preparation of imidacloprid, comprising steps of:

Mixing the imidacloprid technical, antioxidant, alcohol solvent and stabilizer to obtain the external preparation of imidacloprid.

In some embodiments of the present disclosure, the mixing is conducted by:

Heating and mixing the alcohol solvent, antioxidant and imidacloprid technical to obtain a mixed solution;

Adding the stabilizer to the mixed solution to obtain the external preparation of imidacloprid.

In some embodiments of the present disclosure, the temperature of the heating and mixing is 35-40° C.

The present disclosure provides the use of the external preparation of imidacloprid as an agent against ectoparasites.

In some embodiments of the present disclosure, the ectoparasites are one or more of lice, fleas and ticks.

In some embodiments of the present disclosure, the external preparation of imidacloprid is used for pets.

The present disclosure provides an external preparation of imidacloprid, including the following components in percentage by mass: 5-15% of imidacloprid technical; 0.05-0.2% of antioxidant; 80-90% of alcohol solvent; 4.95-10% of stabilizer; the antioxidant is one or more of dibutyl hydroxytoluene, hydroxymethyl, propyl hydroxybenzoate and butylated hydroxyanisole; the stabilizer is one or more of propylene carbonate, povidone and copovidone. The present disclosure uses alcohol solvent as the dispersion medium of imidacloprid technical, which can ensure the stability of imidacloprid technical; The present disclosure uses one or more of dibutyl hydroxytoluene, hydroxymethyl, propyl hydroxybenzoate and butylated hydroxyanisole as the antioxidant, which can effectively prevent the oxidation of imidacloprid technical and prolong the preservation time of external preparations of imidacloprid; The present disclosure uses one or more of propylene carbonate, povidone and copovidone as the stabilizer, which can reduce the volatility of the solvent and maintain the stability of the dispersion system. At the same time, the adjuvants used in the present disclosure have the advantages of safety, no skin irritation, low cost, and are suitable for market promotion and application. The external preparation of imidacloprid in the examples of the present disclosure is subjected to skin irritation tests in rabbit, sensitization tests in guinea pig, influencing factors tests, accelerated tests, and long-term tests. The results show that the external preparation of imidacloprid provided by the present disclosure has good safety and high stability; Through the clinical efficacy test of natural cases of dogs and cats infected with lice and fleas, the results show that the external preparation of imidacloprid provided by the present disclosure has better safety and effectiveness in pet clinics.

### BRIEF DESCRIPTION OF THE DRAWINGS

FIG. 1 shows the results of skin irritation test on rabbit;

FIG. 2 shows the results of skin sensitization test on guinea pig.

### DETAILED DESCRIPTION OF THE EMBODIMENTS

The present disclosure provides an external preparation of imidacloprid, including the following components in percentage by mass:

Imidacloprid technical 5-15%;

Antioxidant 0.05-0.2%;

Alcohol solvent 80-90%;

Stabilizer 4.95-10%;

The antioxidant is one or more of dibutyl hydroxytoluene, hydroxymethyl, propyl hydroxybenzoate and butylated hydroxyanisole;

The stabilizer is one or more of propylene carbonate, povidone and copovidone.

Unless otherwise specified, the sources of the raw materials used in the present disclosure are all commercially available.

In terms of mass percentage, the external preparation of imidacloprid provided by the present disclosure includes 5-15% of the imidacloprid technical, preferably 8-12%, more preferably 9-10%. In the present disclosure, the structural formula of imidacloprid is shown in formula 1:

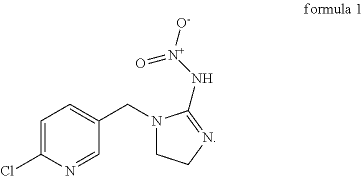 [SMILES: O=[N+]([O-])NC1=NCCN1Cc1ccc(Cl)nc1]

In the present disclosure, the purity of the imidacloprid technical is preferably more than or equal to 99.5%.

In terms of mass percentage, the external preparation of imidacloprid provided by the present disclosure includes 0.05-0.2% of antioxidant, preferably 0.1-0.15%. In the present disclosure, the antioxidant is one or more of dibutyl hydroxytoluene, hydroxymethyl, propyl hydroxybenzoate and butylated hydroxyanisole. In the present disclosure, dibutyl hydroxytoluene (BHT) is used as an antioxidant, which can react with the chain-propagation free radicals in auto-oxidation to destroy the free radicals, thereby interrupting the chain reaction. Dibutyl hydroxytoluene (BHT) can be used as a hydrogen donor and a free radical trapping agent in the antioxidant process. Since there are two powerful electron-pushing groups at positions 2 and 6, dibutyl hydroxytoluene (BHT) has a strong antioxidant effect. Hydroxymethyl, propyl hydroxybenzoate and butylated hydroxyanisole can absorb free radicals generated by oxidation and block the chain reaction of free radicals. The present disclosure uses one or more of dibutyl hydroxytoluene, hydroxymethyl, propyl hydroxybenzoate and butylated hydroxyanisole as the antioxidant, which can effectively prevent the oxidation of the imidacloprid technical and prolong the preservation time of the external preparation of imidacloprid.

In terms of mass percentage, the external preparation of imidacloprid provided by the present disclosure includes 80-90% of alcohol solvents, preferably 82-88%, more preferably 84-86%. In the present disclosure, the alcohol solvent is preferably one or more of benzyl alcohol, propylene glycol and isopropanol.

In terms of mass percentage, the external preparation of imidacloprid provided by the present disclosure includes 4.95-10% of stabilizer, preferably 6-8%. In the present disclosure, the stabilizer is one or more of propylene carbonate, povidone and copovidone. In the present disclosure, the povidone is preferably one or more of povidone K12, povidone K17 and povidone K30, and the copovidone is preferably a linear copolymer of N-vinylpyrrolidone (NVP) and vinyl acetate (VA). The present disclosure uses one or more of propylene carbonate, povidone and copovidone as the stabilizer, which can reduce the volatility of the solvent and maintain the stability of the dispersion system.

In the present disclosure, the dosage form of the external preparation of imidacloprid is preferably a drop. In the present disclosure, the specification of a single external preparation of imidacloprid preferably includes 0.4 mL, 0.8 mL, 1.0 mL, 2.5 mL, and 4.0 mL.

The present disclosure provides a method for preparing the external preparation of imidacloprid, comprising steps of:

Mixing the imidacloprid technical, antioxidant, alcohol solvent and stabilizer to obtain the external preparation of imidacloprid.

In the present disclosure, the mixing is preferably conducted by:

Heating and mixing the alcohol solvent, antioxidant and imidacloprid technical to obtain a mixed solution;

Adding the stabilizer to the mixed solution to obtain the external preparation of imidacloprid.

In the present disclosure, the temperature of the heating and mixing is preferably 35-40° C., more preferably 36-38° C. In the present disclosure, the heating and mixing are preferably performed under stirring conditions. In the present disclosure, the alcohol solvent is preferably added first, then the antioxidant is added, and then the imidacloprid technical is added. In the present disclosure, there is no special requirement for the heating and mixing time, as long as the above raw materials are stirred uniformly.

After the mixed solution is obtained, the present disclosure uses a stabilizer to fix the volume of the mixed solution to obtain the external preparation of imidacloprid.

After the external preparation of imidacloprid is obtained, the present disclosure preferably conducts inspecting and packaging.

In the present disclosure, the material for packaging is preferably polyethylene.

The present disclosure provides the use of the external preparation of imidacloprid as an agent against ectoparasites.

In the present disclosure, the ectoparasites are one or more of lice, fleas and ticks.

In the present disclosure, the external preparation of imidacloprid is preferably used for pets, and the dosage form is preferably drops. In the present disclosure, the pet is preferably a dog and/or cat. In the present disclosure, when the external preparation of imidacloprid is used for dogs, the specification preferably includes 0.4 mL, 1.0 mL, 2.5 mL, and 4.0 mL; when the external preparation of imidacloprid is used for cats, the specification preferably includes 0.4 mL and 0.8 mL.

In the present disclosure, the method of using the external preparation of imidacloprid preferably includes steps of:

Applying the external preparation of imidacloprid to the surface of pet skin or hair, and applying once can achieve the effective effect on ectoparasites for 4 weeks.

The external preparation of imidacloprid provided by the present disclosure and the preparation method and use thereof will be described in detail below in conjunction with examples, but they should not be understood as limiting the protection scope of the present disclosure.

### EXAMPLE 1

The external preparation of imidacloprid was prepared according to the formula in Table 1. The method is as follows:

(1) A prescription amount of alcohol solvent was taken and heated to 40° C.;

(2) When the temperature was heated to 40° C., the antioxidant was added thereto and stirred until completely dissolved;

(3) The temperature was kept at 40° C., the prescription amount of imidacloprid was added, stirred until completely dissolved, and then cooled to room temperature;

(4) The volume was fixed to the prescription amount with stabilizer, and the resulting product was inspected;

(5) The product was packed with a medicinal polyethylene composite ointment tube after passing the inspection, and the finished product was stored after passing the inspection.

### EXAMPLE 2-6

The formulas of Examples 2-6 are shown in Table 1, and the preparation method is the same as that of Example 1.

TABLE 1
The amount of raw materials used in Examples 1-6

| | type and amount (%) of raw materials | | | | | |
|---|---|---|---|---|---|---|
| | Example 1 | Example 2 | Example 3 | Example 4 | Example 5 | Example 6 |
| imidacloprid | 10 | 10 | 10 | 10 | 10 | 10 |
| antioxidant | dibutyl | hydroxymethyl | propyl | butylated | dibutyl | hydroxymethyl |
| | hydroxytoluene | 0.09 | hydroxybenzoate | hydroxyanisole | hydroxytoluene | 0.08 |
| | 0.09 | | 0.09 | 0.08 | 0.08 | |
| alcohol | benzyl | propylene | isopropanol | benzyl | propylene | isopropanol |
| solvent | alcohol | glycol | 80.91 | alcohol | glycol | 80.92 |
| | 81.91 | 84.91 | | 81.92 | 84.92 | |
| stabilizer | propylene | povidone | copovidone | propylene | povidone | propylene |
| | carbonate | K12 | 9 | carbonate | K17 | carbonate |
| | 8 | 5 | | 8 | 5 | 9 |

Performance Testing

(1) Influencing Factors Test

The specifications of the external preparation of imidacloprid in Example 1 were divided into 0.4 mL/for dogs, 1.0 mL/for dogs, 2.5 mL/for dogs, 4.0 mL/for dogs, 0.4 mL/for cats, and 0.8 mL/for cats, and subjected to strong light irradiation test (irradiation intensity of 45001x±5001x), high temperature (60° C.) test, high humidity (25° C., relative humidity of 90%) test in accordance with the “Technical Guidelines for Stability Test of Veterinary Drugs” in the 2015 edition of the “Chinese Veterinary Pharmacopoeia”, test results are shown in Table 2.

TABLE 2
Results of influencing factors test of imidacloprid drops in Example 1

| | | | | | dibutyl | | imidacloprid |
|---|---|---|---|---|---|---|---|
| | | | | relative | hydroxytoluene | | content |
| | batch No. | factor | appearance | density | (%) | moisture | (%) |
| 0.4 mL/ | 20191201 | 0 d | yellow to | 1.102 | 0.09 | 0.1 | 100.9 |
| for dogs | | | light brown | | | | |
| | | | clear liquid | | | | |
| | | light | yellow to | 1.100 | 0.08 | 0.1 | 100.9 |
| | | irradiation | light brown | | | | |
| | | for 5 d | clear liquid | | | | |
| | | light | yellow to | 1.101 | 0.09 | 0.1 | 100.7 |
| | | irradiation | light brown | | | | |
| | | for 10 d | clear liquid | | | | |
| | | high | yellow to | 1.101 | 0.09 | 0.1 | 100.6 |
| | | temperature | light brown | | | | |
| | | for 5 d | clear liquid | | | | |
| | | high | yellow to | 1.100 | 0.09 | 0.1 | 100.5 |
| | | temperature | light brown | | | | |
| | | for 10 d | clear liquid | | | | |
| | | RH 75% | yellow to | 1.101 | 0.09 | 0.1 | 100.7 |
| | | for 5 d | light brown | | | | |
| | | | clear liquid | | | | |
| | | RH 75% | yellow to | 1.101 | 0.09 | 0.1 | 100.3 |
| | | for 10 d | light brown | | | | |
| | | | clear liquid | | | | |
| 1.0 mL/ | 20191201 | 0 d | yellow to | 1.101 | 0.09 | 0.1 | 100.8 |
| for dogs | | | light brown | | | | |
| | | | clear liquid | | | | |
| | | light | yellow to | 1.101 | 0.09 | 0.1 | 100.7 |
| | | irradiation | light brown | | | | |
| | | for 5 d | clear liquid | | | | |
| | | light | yellow to | 1.102 | 0.09 | 0.1 | 100.6 |
| | | irradiation | light brown | | | | |
| | | for 10 d | clear liquid | | | | |
| | | high | yellow to | 1.101 | 0.09 | 0.1 | 100.7 |
| | | temperature | light brown | | | | |
| | | for 5 d | clear liquid | | | | |
| | | high | yellow to | 1.101 | 0.09 | 0.1 | 100.4 |
| | | temperature | light brown | | | | |
| | | for 10 d | clear liquid | | | | |
| | | RH 75% | yellow to | 1.101 | 0.09 | 0.1 | 100.7 |
| | | for 5 d | light brown | | | | |
| | | | clear liquid | | | | |
| | | RH 75% | yellow to | 1.101 | 0.09 | 0.1 | 100.2 |
| | | for 10 d | light brown | | | | |
| | | | clear liquid | | | | |
| 2.5 mL/ | 20191201 | 0 d | yellow to | 1.101 | 0.09 | 0.1 | 101.0 |
| for dogs | | | light brown | | | | |
| | | | clear liquid | | | | |
| | | light | yellow to | 1.102 | 0.09 | 0.1 | 100.9 |
| | | irradiation | light brown | | | | |
| | | for 5 d | clear liquid | | | | |
| | | light | yellow to | 1.101 | 0.09 | 0.1 | 100.7 |
| | | irradiation | light brown | | | | |
| | | for 10 d | clear liquid | | | | |
| | | high | yellow to | 1.101 | 0.09 | 0.1 | 100.8 |
| | | temperature | light brown | | | | |
| | | for 5 d | clear liquid | | | | |
| | | high | yellow to | 1.101 | 0.09 | 0.1 | 100.8 |
| | | temperature | light brown | | | | |
| | | for 10 d | clear liquid | | | | |
| | | RH 90% | yellow to | 1.101 | 0.09 | 0.1 | 100.9 |
| | | for 5 d | light brown | | | | |
| | | | clear liquid | | | | |
| | | RH 90% | yellow to | 1.101 | 0.09 | 0.1 | 101.0 |
| | | for 10 d | light brown | | | | |
| | | | clear liquid | | | | |
| 4.0 mL/ | 20191201 | 0 d | yellow to | 1.101 | 0.08 | 0.1 | 100.8 |
| for dogs | | | light brown | | | | |
| | | | clear liquid | | | | |
| | | light | yellow to | 1.101 | 0.08 | 0.1 | 100.7 |
| | | irradiation | light brown | | | | |
| | | for 5 d | clear liquid | | | | |
| | | light | yellow to | 1.102 | 0.08 | 0.1 | 100.7 |
| | | irradiation | light brown | | | | |
| | | for 10 d | clear liquid | | | | |
| | | high | yellow to | 1.102 | 0.08 | 0.1 | 100.8 |
| | | temperature | light brown | | | | |
| | | for 5 d | clear liquid | | | | |
| | | high | yellow to | 1.100 | 0.08 | 0.1 | 100.6 |
| | | temperature | light brown | | | | |
| | | for 10 d | clear liquid | | | | |
| | | RH 90% | yellow to | 1.101 | 0.08 | 0.1 | 100.6 |
| | | for 5 d | light brown | | | | |
| | | | clear liquid | | | | |
| | | RH 90% | yellow to | 1.101 | 0.08 | 0.1 | 100.4 |
| | | for 10 d | light brown | | | | |
| | | | clear liquid | | | | |
| 0.4 mL/ | 20200101 | 0 d | yellow to | 1.100 | 0.08 | 0.1 | 101.8 |
| for cats | | | light brown | | | | |
| | | | clear liquid | | | | |
| | | light | yellow to | 1.102 | 0.08 | 0.1 | 101.4 |
| | | irradiation | light brown | | | | |
| | | for 5 d | clear liquid | | | | |
| | | light | yellow to | 1.101 | 0.08 | 0.1 | 101.1 |
| | | irradiation | light brown | | | | |
| | | for 10 d | clear liquid | | | | |
| | | high | yellow to | 1.101 | 0.08 | 0.1 | 101.1 |
| | | temperature | light brown | | | | |
| | | for 5 d | clear liquid | | | | |
| | | high | yellow to | 1.100 | 0.08 | 0.1 | 100.9 |
| | | temperature | light brown | | | | |
| | | for 10 d | clear liquid | | | | |
| | | RH 90% | yellow to | 1.101 | 0.08 | 0.1 | 101.3 |
| | | for 5 d | light brown | | | | |
| | | | clear liquid | | | | |
| | | RH 90% | yellow to | 1.101 | 0.08 | 0.1 | 101.1 |
| | | for 10 d | light brown | | | | |
| | | | clear liquid | | | | |
| 0.8 mL/ | 20200101 | 0 d | yellow to | 1.102 | 0.08 | 0.1 | 101.1 |
| for cats | | | light brown | | | | |
| | | | clear liquid | | | | |
| | | light | yellow to | 1.101 | 0.08 | 0.1 | 101.1 |
| | | irradiation | light brown | | | | |
| | | for 5 d | clear liquid | | | | |
| | | light | yellow to | 1.100 | 0.08 | 0.1 | 100.9 |
| | | irradiation | light brown | | | | |
| | | for 10 d | clear liquid | | | | |
| | | high | yellow to | 1.100 | 0.08 | 0.1 | 100.7 |
| | | temperature | light brown | | | | |
| | | for 5 d | clear liquid | | | | |
| | | high | yellow to | 1.101 | 0.08 | 0.1 | 100.5 |
| | | temperature | light brown | | | | |
| | | for 10 d | clear liquid | | | | |
| | | RH 90% | yellow to | 1.101 | 0.08 | 0.1 | 100.9 |
| | | for 5 d | light brown | | | | |
| | | | clear liquid | | | | |
| | | RH 90% | yellow to | 1.101 | 0.08 | 0.1 | 100.7 |
| | | for 10 d | light brown | | | | |
| | | | clear liquid | | | | |

The external preparations of imidacloprid obtained in Examples 2-6 were subjected to influencing factors test according to the above method, and the obtained results are shown in Table 3.

TABLE 3
Results of influencing factors test of imidacloprid drops obtained in Examples 2-6

| | | | | | dibutyl | | imidacloprid |
|---|---|---|---|---|---|---|---|
| | | | | relative | hydroxytoluene | | content |
| | batch No. | factor | appearance | density | (%) | moisture | (%) |
| Example 2 | 20191102 | 0 d | yellow to | 1.101 | 0.09 | 0.1 | 102.1 |
| | | | light brown | | | | |
| | | | clear liquid | | | | |
| | | light | yellow to | 1.102 | 0.09 | 0.1 | 102.4 |
| | | irradiation | light brown | | | | |
| | | for 5 d | clear liquid | | | | |
| | | light | yellow to | 1.101 | 0.09 | 0.1 | 101.9 |
| | | irradiation | light brown | | | | |
| | | for 10 d | clear liquid | | | | |
| | | high | yellow to | 1.101 | 0.09 | 0.1 | 101.8 |
| | | temperature | light brown | | | | |
| | | for 5 d | clear liquid | | | | |
| | | high | yellow to | 1.102 | 0.09 | 0.1 | 101.2 |
| | | temperature | light brown | | | | |
| | | for 10 d | clear liquid | | | | |
| | | RH 75% | yellow to | 1.102 | 0.09 | 0.1 | 101.6 |
| | | for 5 d | light brown | | | | |
| | | | clear liquid | | | | |
| | | RH 75% | yellow to | 1.101 | 0.09 | 0.1 | 102.2 |
| | | for 10 d | light brown | | | | |
| | | | clear liquid | | | | |
| Example 3 | 20191103 | 0 d | yellow to | 1.102 | 0.09 | 0.1 | 101.0 |
| | | | light brown | | | | |
| | | | clear liquid | | | | |
| | | light | yellow to | 1.102 | 0.09 | 0.1 | 100.9 |
| | | irradiation | light brown | | | | |
| | | for 5 d | clear liquid | | | | |
| | | light | yellow to | 1.101 | 0.09 | 0.1 | 100.7 |
| | | irradiation | light brown | | | | |
| | | for 10 d | clear liquid | | | | |
| | | high | yellow to | 1.101 | 0.09 | 0.1 | 101.5 |
| | | temperature | light brown | | | | |
| | | for 5 d | clear liquid | | | | |
| | | high | yellow to | 1.102 | 0.09 | 0.1 | 100.2 |
| | | temperature | light brown | | | | |
| | | for 10 d | clear liquid | | | | |
| | | RH 90% | yellow to | 1.101 | 0.09 | 0.1 | 100.9 |
| | | for 5 d | light brown | | | | |
| | | | clear liquid | | | | |
| | | RH 90% | yellow to | 1.102 | 0.09 | 0.1 | 101.0 |
| | | for 10 d | light brown | | | | |
| | | | clear liquid | | | | |
| Example 4 | 20191201104 | 0 d | yellow to | 1.101 | 0.08 | 0.1 | 102.3 |
| | | | light brown | | | | |
| | | | clear liquid | | | | |
| | | light | yellow to | 1.101 | 0.08 | 0.1 | 101.8 |
| | | irradiation | light brown | | | | |
| | | for 5 d | clear liquid | | | | |
| | | light | yellow to | 1.102 | 0.08 | 0.1 | 101.5 |
| | | irradiation | light brown | | | | |
| | | for 10 d | clear liquid | | | | |
| | | high | yellow to | 1.102 | 0.08 | 0.1 | 101.5 |
| | | temperature | light brown | | | | |
| | | for 5 d | clear liquid | | | | |
| | | high | yellow to | 1.100 | 0.08 | 0.1 | 101.1 |
| | | temperature | light brown | | | | |
| | | for 10 d | clear liquid | | | | |
| | | RH 90% | yellow to | 1.101 | 0.08 | 0.1 | 102.1 |
| | | for 5 d | light brown | | | | |
| | | | clear liquid | | | | |
| | | RH 90% | yellow to | 1.101 | 0.08 | 0.1 | 102.5 |
| | | for 10 d | light brown | | | | |
| | | | clear liquid | | | | |
| Example 5 | 20191105 | 0 d | yellow to | 1.100 | 0.08 | 0.1 | 101.5 |
| | | | light brown | | | | |
| | | | clear liquid | | | | |
| | | light | yellow to | 1.102 | 0.08 | 0.1 | 101.2 |
| | | irradiation | light brown | | | | |
| | | for 5 d | clear liquid | | | | |
| | | light | yellow to | 1.101 | 0.08 | 0.1 | 101.1 |
| | | irradiation | light brown | | | | |
| | | for 10 d | clear liquid | | | | |
| | | high | yellow to | 1.101 | 0.08 | 0.1 | 100.8 |
| | | temperature | light brown | | | | |
| | | for 5 d | clear liquid | | | | |
| | | high | yellow to | 1.100 | 0.08 | 0.1 | 100.1 |
| | | temperature | light brown | | | | |
| | | for 10 d | clear liquid | | | | |
| | | RH 90% | yellow to | 1.101 | 0.08 | 0.1 | 101.3 |
| | | for 5 d | light brown | | | | |
| | | | clear liquid | | | | |
| | | RH 90% | yellow to | 1.101 | 0.08 | 0.1 | 101.4 |
| | | for 10 d | light brown | | | | |
| | | | clear liquid | | | | |
| Example 6 | 20191106 | 0 d | yellow to | 1.102 | 0.08 | 0.1 | 102.6 |
| | | | light brown | | | | |
| | | | clear liquid | | | | |
| | | light | yellow to | 1.101 | 0.08 | 0.1 | 102.2 |
| | | irradiation | light brown | | | | |
| | | for 5 d | clear liquid | | | | |
| | | light | yellow to | 1.100 | 0.08 | 0.1 | 102.3 |
| | | irradiation | light brown | | | | |
| | | for 10 d | clear liquid | | | | |
| | | high | yellow to | 1.100 | 0.08 | 0.1 | 101.8 |
| | | temperature | light brown | | | | |
| | | for 5 d | clear liquid | | | | |
| | | high | yellow to | 1.101 | 0.08 | 0.1 | 101.4 |
| | | temperature | light brown | | | | |
| | | for 10 d | clear liquid | | | | |
| | | RH 90% | yellow to | 1.101 | 0.08 | 0.1 | 102.4 |
| | | for 5 d | light brown | | | | |
| | | | clear liquid | | | | |
| | | RH 90% | yellow to | 1.101 | 0.08 | 0.1 | 102.5 |
| | | for 10 d | light brown | | | | |
| | | | clear liquid | | | | |

The above results show that after 10 days, the appearance, relative density, antioxidant content, imidacloprid content, and moisture of the imidacloprid drops have no significant changes.

(2) Long-Term Test

The preparations prepared in the Example 1 were sampled regularly at 0, 3, 6, 9, 12, and 18 months, and the indicators were tested under the test conditions of 25° C.±2° C. and RH 60%±10% in accordance with the “Guiding Principles for Stability Testing of Veterinary Drugs” in the 2015 edition of the “Chinese Veterinary Pharmacopoeia”. The results are shown in Table 3.

TABLE 3
Long-term test results

| | | time/ | | relative | dibutyl | | content |
|---|---|---|---|---|---|---|---|
| | batch No. | month | appearance | density | hydroxytoluene | moisture | (%) |
| 0.4 mL/ | 20191201 | 0 | yellow to | 1.102 | 0.09 | 0.1 | 100.0 |
| for dogs | | | light brown | | | | |
| | | | clear liquid | | | | |
| | | 3 | yellow to | 1.100 | 0.09 | 0.1 | 99.4 |
| | | | light brown | | | | |
| | | | clear liquid | | | | |
| | | 6 | yellow to | 1.099 | 0.09 | 0.1 | 99.3 |
| | | | light brown | | | | |
| | | | clear liquid | | | | |
| | | 9 | yellow to | 1.101 | 0.08 | 0.1 | 99.7 |
| | | | light brown | | | | |
| | | | clear liquid | | | | |
| | | 12 | yellow to | 1.101 | 0.09 | 0.1 | 99.7 |
| | | | light brown | | | | |
| | | | clear liquid | | | | |
| | | 18 | yellow to | 1.101 | 0.08 | 0.1 | 99.1 |
| | | | light brown | | | | |
| | | | clear liquid | | | | |
| | 20191202 | 0 | yellow to | 1.101 | 0.09 | 0.1 | 99.4 |
| | | | light brown | | | | |
| | | | clear liquid | | | | |
| | | 3 | yellow to | 1.101 | 0.09 | 0.1 | 99.1 |
| | | | light brown | | | | |
| | | | clear liquid | | | | |
| | | 6 | yellow to | 1.101 | 0.08 | 0.1 | 98.8 |
| | | | light brown | | | | |
| | | | clear liquid | | | | |
| | | 9 | yellow to | 1.099 | 0.09 | 0.1 | 100.2 |
| | | | light brown | | | | |
| | | | clear liquid | | | | |
| | | 12 | yellow to | 1.099 | 0.09 | 0.1 | 99.1 |
| | | | light brown | | | | |
| | | | clear liquid | | | | |
| | | 18 | yellow to | 1.100 | 0.09 | 0.1 | 99.3 |
| | | | light brown | | | | |
| | | | clear liquid | | | | |
| | 20191203 | 0 | yellow to | 1.100 | 0.09 | 0.1 | 99.8 |
| | | | light brown | | | | |
| | | | clear liquid | | | | |
| | | 3 | yellow to | 1.100 | 0.09 | 0.1 | 99.6 |
| | | | light brown | | | | |
| | | | clear liquid | | | | |
| | | 6 | yellow to | 1.099 | 0.09 | 0.1 | 100.5 |
| | | | light brown | | | | |
| | | | clear liquid | | | | |
| | | 9 | yellow to | 1.099 | 0.09 | 0.1 | 99.8 |
| | | | light brown | | | | |
| | | | clear liquid | | | | |
| | | 12 | yellow to | 1.101 | 0.09 | 0.1 | 99.2 |
| | | | light brown | | | | |
| | | | clear liquid | | | | |
| | | 18 | yellow to | 1.101 | 0.09 | 0.1 | 99.4 |
| | | | light brown | | | | |
| | | | clear liquid | | | | |
| 1.0 mL/ | 20191201 | 0 | yellow to | 1.100 | 0.09 | 0.1 | 99.9 |
| for dogs | | | light brown | | | | |
| | | | clear liquid | | | | |
| | | 3 | yellow to | 1.101 | 0.09 | 0.1 | 99.4 |
| | | | light brown | | | | |
| | | | clear liquid | | | | |
| | | 6 | yellow to | 1.101 | 0.09 | 0.1 | 100.2 |
| | | | light brown | | | | |
| | | | clear liquid | | | | |
| | | 9 | yellow to | 1.100 | 0.09 | 0.1 | 99.8 |
| | | | light brown | | | | |
| | | | clear liquid | | | | |
| | | 12 | yellow to | 1.099 | 0.08 | 0.1 | 99.1 |
| | | | light brown | | | | |
| | | | clear liquid | | | | |
| | | 18 | yellow to | 1.099 | 0.09 | 0.1 | 99.4 |
| | | | light brown | | | | |
| | | | clear liquid | | | | |
| | 20191202 | 0 | yellow to | 1.101 | 0.09 | 0.1 | 100.1 |
| | | | light brown | | | | |
| | | | clear liquid | | | | |
| | | 3 | yellow to | 1.100 | 0.09 | 0.1 | 100.4 |
| | | | light brown | | | | |
| | | | clear liquid | | | | |
| | | 6 | yellow to | 1.100 | 0.09 | 0.1 | 99.8 |
| | | | light brown | | | | |
| | | | clear liquid | | | | |
| | | 9 | yellow to | 1.100 | 0.09 | 0.1 | 99.2 |
| | | | light brown | | | | |
| | | | clear liquid | | | | |
| | | 12 | yellow to | 1.101 | 0.09 | 0.1 | 99.3 |
| | | | light brown | | | | |
| | | | clear liquid | | | | |
| | | 18 | yellow to | 1.101 | 0.09 | 0.1 | 99.8 |
| | | | light brown | | | | |
| | | | clear liquid | | | | |
| | 20191203 | 0 | yellow to | 1.102 | 0.09 | 0.1 | 99.5 |
| | | | light brown | | | | |
| | | | clear liquid | | | | |
| | | 3 | yellow to | 1.101 | 0.08 | 0.1 | 98.8 |
| | | | light brown | | | | |
| | | | clear liquid | | | | |
| | | 6 | yellow to | 1.101 | 0.09 | 0.1 | 99.6 |
| | | | light brown | | | | |
| | | | clear liquid | | | | |
| | | 9 | yellow to | 1.100 | 0.09 | 0.1 | 98.9 |
| | | | light brown | | | | |
| | | | clear liquid | | | | |
| | | 12 | yellow to | 1.099 | 0.09 | 0.1 | 99.1 |
| | | | light brown | | | | |
| | | | clear liquid | | | | |
| | | 18 | yellow to | 1.099 | 0.09 | 0.1 | 98.5 |
| | | | light brown | | | | |
| | | | clear liquid | | | | |
| 2.5 mL/ | 20191201 | 0 | yellow to | 1.102 | 0.09 | 0.1 | 99.5 |
| for dogs | | | light brown | | | | |
| | | | clear liquid | | | | |
| | | 3 | yellow to | 1.100 | 0.09 | 0.1 | 99.1 |
| | | | light brown | | | | |
| | | | clear liquid | | | | |
| | | 6 | yellow to | 1.100 | 0.09 | 0.1 | 98.9 |
| | | | light brown | | | | |
| | | | clear liquid | | | | |
| | | 9 | yellow to | 1.101 | 0.09 | 0.1 | 99.0 |
| | | | light brown | | | | |
| | | | clear liquid | | | | |
| | | 12 | yellow to | 1.101 | 0.09 | 0.1 | 99.1 |
| | | | light brown | | | | |
| | | | clear liquid | | | | |
| | | 18 | yellow to | 1.101 | 0.09 | 0.1 | 98.3 |
| | | | light brown | | | | |
| | | | clear liquid | | | | |
| | 20191202 | 0 | yellow to | 1.102 | 0.09 | 0.1 | 99.0 |
| | | | light brown | | | | |
| | | | clear liquid | | | | |
| | | 3 | yellow to | 1.101 | 0.09 | 0.1 | 98.3 |
| | | | light brown | | | | |
| | | | clear liquid | | | | |
| | | 6 | yellow to | 1.101 | 0.09 | 0.1 | 98.8 |
| | | | light brown | | | | |
| | | | clear liquid | | | | |
| | | 9 | yellow to | 1.100 | 0.09 | 0.1 | 99.3 |
| | | | light brown | | | | |
| | | | clear liquid | | | | |
| | | 12 | yellow to | 1.100 | 0.09 | 0.1 | 98.5 |
| | | | light brown | | | | |
| | | | clear liquid | | | | |
| | | 18 | yellow to | 1.099 | 0.09 | 0.1 | 98.3 |
| | | | light brown | | | | |
| | | | clear liquid | | | | |
| | 20191203 | 0 | yellow to | 1.101 | 0.09 | 0.1 | 99.1 |
| | | | light brown | | | | |
| | | | clear liquid | | | | |
| | | 3 | yellow to | 1.100 | 0.09 | 0.1 | 98.7 |
| | | | light brown | | | | |
| | | | clear liquid | | | | |
| | | 6 | yellow to | 1.100 | 0.09 | 0.1 | 98.9 |
| | | | light brown | | | | |
| | | | clear liquid | | | | |
| | | 9 | yellow to | 1.101 | 0.09 | 0.1 | 98.3 |
| | | | light brown | | | | |
| | | | clear liquid | | | | |
| | | 12 | yellow to | 1.101 | 0.09 | 0.1 | 98.8 |
| | | | light brown | | | | |
| | | | clear liquid | | | | |
| | | 18 | yellow to | 1.101 | 0.09 | 0.1 | 98.3 |
| | | | light brown | | | | |
| | | | clear liquid | | | | |
| 4.0 mL/ | 20191201 | 0 | yellow to | 1.101 | 0.08 | 0.1 | 99.4 |
| for dogs | | | light brown | | | | |
| | | | clear liquid | | | | |
| | | 3 | yellow to | 1.101 | 0.08 | 0.1 | 99.1 |
| | | | light brown | | | | |
| | | | clear liquid | | | | |
| | | 6 | yellow to | 1.099 | 0.08 | 0.1 | 99.3 |
| | | | light brown | | | | |
| | | | clear liquid | | | | |
| | | 9 | yellow to | 1.099 | 0.08 | 0.1 | 98.9 |
| | | | light brown | | | | |
| | | | clear liquid | | | | |
| | | 12 | yellow to | 1.100 | 0.08 | 0.1 | 98.4 |
| | | | light brown | | | | |
| | | | clear liquid | | | | |
| | | 18 | yellow to | 1.100 | 0.08 | 0.1 | 98.9 |
| | | | light brown | | | | |
| | | | clear liquid | | | | |
| | 20191202 | 0 | yellow to | 1.102 | 0.08 | 0.1 | 99.1 |
| | | | light brown | | | | |
| | | | clear liquid | | | | |
| | | 3 | yellow to | 1.100 | 0.08 | 0.1 | 98.8 |
| | | | light brown | | | | |
| | | | clear liquid | | | | |
| | | 6 | yellow to | 1.100 | 0.08 | 0.1 | 98.6 |
| | | | light brown | | | | |
| | | | clear liquid | | | | |
| | | 9 | yellow to | 1.101 | 0.08 | 0.1 | 98.9 |
| | | | light brown | | | | |
| | | | clear liquid | | | | |
| | | 12 | yellow to | 1.101 | 0.08 | 0.1 | 98.8 |
| | | | light brown | | | | |
| | | | clear liquid | | | | |
| | | 18 | yellow to | 1.101 | 0.08 | 0.1 | 98.2 |
| | | | light brown | | | | |
| | | | clear liquid | | | | |
| | 20191203 | 0 | yellow to | 1.100 | 0.08 | 0.1 | 99.0 |
| | | | light brown | | | | |
| | | | clear liquid | | | | |
| | | 3 | yellow to | 1.101 | 0.08 | 0.1 | 98.5 |
| | | | light brown | | | | |
| | | | clear liquid | | | | |
| | | 6 | yellow to | 1.101 | 0.08 | 0.1 | 98.8 |
| | | | light brown | | | | |
| | | | clear liquid | | | | |
| | | 9 | yellow to | 1.100 | 0.08 | 0.1 | 99.3 |
| | | | light brown | | | | |
| | | | clear liquid | | | | |
| | | 12 | yellow to | 1.100 | 0.08 | 0.1 | 98.7 |
| | | | light brown | | | | |
| | | | clear liquid | | | | |
| | | 18 | yellow to | 1.100 | 0.08 | 0.1 | 98.3 |
| | | | light brown | | | | |
| | | | clear liquid | | | | |
| 0.4 mL/ | 20200101 | 0 | yellow to | 1.100 | 0.08 | 0.1 | 99.0 |
| for cats | | | light brown | | | | |
| | | | clear liquid | | | | |
| | | 3 | yellow to | 1.100 | 0.08 | 0.1 | 98.5 |
| | | | light brown | | | | |
| | | | clear liquid | | | | |
| | | 6 | yellow to | 1.100 | 0.08 | 0.1 | 98.8 |
| | | | light brown | | | | |
| | | | clear liquid | | | | |
| | | 9 | yellow to | 1.101 | 0.08 | 0.1 | 98.2 |
| | | | light brown | | | | |
| | | | clear liquid | | | | |
| | | 12 | yellow to | 1.101 | 0.08 | 0.1 | 99.2 |
| | | | light brown | | | | |
| | | | clear liquid | | | | |
| | | 18 | yellow to | 1.101 | 0.08 | 0.1 | 98.7 |
| | | | light brown | | | | |
| | | | clear liquid | | | | |
| | 20200102 | 0 | yellow to | 1.101 | 0.08 | 0.1 | 98.8 |
| | | | light brown | | | | |
| | | | clear liquid | | | | |
| | | 3 | yellow to | 1.101 | 0.08 | 0.1 | 98.2 |
| | | | light brown | | | | |
| | | | clear liquid | | | | |
| | | 6 | yellow to | 1.101 | 0.08 | 0.1 | 99.0 |
| | | | light brown | | | | |
| | | | clear liquid | | | | |
| | | 9 | yellow to | 1.100 | 0.08 | 0.1 | 99.3 |
| | | | light brown | | | | |
| | | | clear liquid | | | | |
| | | 12 | yellow to | 1.100 | 0.08 | 0.1 | 98.1 |
| | | | light brown | | | | |
| | | | clear liquid | | | | |
| | | 18 | yellow to | 1.100 | 0.08 | 0.1 | 98.5 |
| | | | light brown | | | | |
| | | | clear liquid | | | | |
| | 20200103 | 0 | yellow to | 1.101 | 0.08 | 0.1 | 98.4 |
| | | | light brown | | | | |
| | | | clear liquid | | | | |
| | | 3 | yellow to | 1.100 | 0.08 | 0.1 | 98.7 |
| | | | light brown | | | | |
| | | | clear liquid | | | | |
| | | 6 | yellow to | 1.100 | 0.08 | 0.1 | 98.1 |
| | | | light brown | | | | |
| | | | clear liquid | | | | |
| | | 9 | yellow to | 1.100 | 0.08 | 0.1 | 98.7 |
| | | | light brown | | | | |
| | | | clear liquid | | | | |
| | | 12 | yellow to | 1.100 | 0.08 | 0.1 | 98.3 |
| | | | light brown | | | | |
| | | | clear liquid | | | | |
| | | 18 | yellow to | 1.100 | 0.08 | 0.1 | 98.0 |
| | | | light brown | | | | |
| | | | clear liquid | | | | |
| 0.8 mL/ | 20200101 | 0 | yellow to | 1.102 | 0.08 | 0.1 | 98.5 |
| for cats | | | light brown | | | | |
| | | | clear liquid | | | | |
| | | 3 | yellow to | 1.101 | 0.08 | 0.1 | 98.1 |
| | | | light brown | | | | |
| | | | clear liquid | | | | |
| | | 6 | yellow to | 1.101 | 0.08 | 0.1 | 98.8 |
| | | | light brown | | | | |
| | | | clear liquid | | | | |
| | | 9 | yellow to | 1.101 | 0.08 | 0.1 | 99.1 |
| | | | light brown | | | | |
| | | | clear liquid | | | | |
| | | 12 | yellow to | 1.100 | 0.08 | 0.1 | 98.6 |
| | | | light brown | | | | |
| | | | clear liquid | | | | |
| | | 18 | yellow to | 1.100 | 0.08 | 0.1 | 98.2 |
| | | | light brown | | | | |
| | | | clear liquid | | | | |
| | 20200102 | 0 | yellow to | 1.101 | 0.08 | 0.1 | 98.7 |
| | | | light brown | | | | |
| | | | clear liquid | | | | |
| | | 3 | yellow to | 1.100 | 0.08 | 0.1 | 98.3 |
| | | | light brown | | | | |
| | | | clear liquid | | | | |
| | | 6 | yellow to | 1.100 | 0.08 | 0.1 | 98.0 |
| | | | light brown | | | | |
| | | | clear liquid | | | | |
| | | 9 | yellow to | 1.100 | 0.08 | 0.1 | 98.8 |
| | | | light brown | | | | |
| | | | clear liquid | | | | |
| | | 12 | yellow to | 1.101 | 0.08 | 0.1 | 99.1 |
| | | | light brown | | | | |
| | | | clear liquid | | | | |
| | | 18 | yellow to | 1.101 | 0.08 | 0.1 | 98.2 |
| | | | light brown | | | | |
| | | | clear liquid | | | | |
| | 20200203 | 0 | yellow to | 1.102 | 0.08 | 0.1 | 98.9 |
| | | | light brown | | | | |
| | | | clear liquid | | | | |
| | | 3 | yellow to | 1.101 | 0.08 | 0.1 | 99.2 |
| | | | light brown | | | | |
| | | | clear liquid | | | | |
| | | 6 | yellow to | 1.101 | 0.08 | 0.1 | 98.3 |
| | | | light brown | | | | |
| | | | clear liquid | | | | |
| | | 9 | yellow to | 1.100 | 0.08 | 0.1 | 99.3 |
| | | | light brown | | | | |
| | | | clear liquid | | | | |
| | | 12 | yellow to | 1.100 | 0.08 | 0.1 | 99.6 |
| | | | light brown | | | | |
| | | | clear liquid | | | | |
| | | 18 | yellow to | 1.100 | 0.08 | 0.1 | 98.4 |
| | | | light brown | | | | |
| | | | clear liquid | | | | |

The results show that the external preparation of imidacloprid was placed under the conditions of 25° C.±2° C., RH 60%±10% for 18 months, the appearance of the preparation is always yellow to light brown clear liquid; the content of dibutyl hydroxytoluene has not obvious changed, the content change of the active ingredient imidacloprid is less than 1%, which is not significant; other quality indicators are in compliance with the regulations, indicating that the preparation has good stability under long-term conditions. During the long-term test, the appearance, content, relative density and other indicators of the drug packaged by the medicinal polyethylene composite ointment tube are stable, which prove that the packaging material has no effect on the quality of the drug.

(3) Skin Sensitization Test

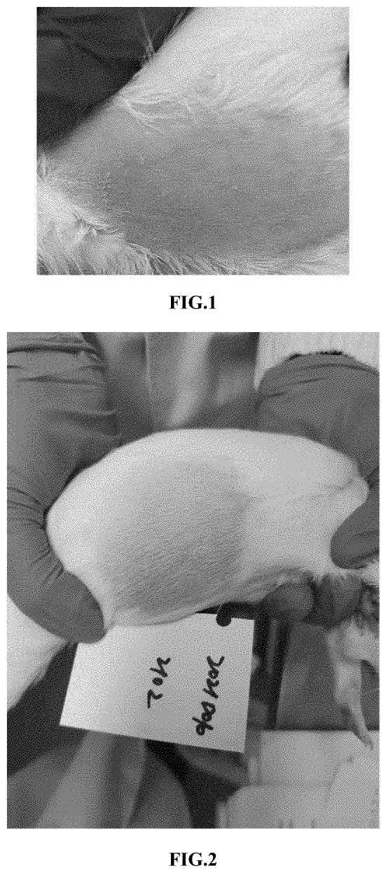

The external preparations of imidacloprid prepared in the above examples were commissioned to Hubei Provincial Academy of Preventive Medicine to conduct a single skin irritation test on rabbit and skin sensitization test on guinea pig using the prepared imidacloprid drops in accordance with the “Quality Management Regulations for Non-clinical Research of Veterinary Drugs” (GLP) of the Ministry of Agriculture and Rural Affairs of China, the results of a single skin irritation test on rabbits are shown in FIG. 1, and the results of a skin sensitization test on guinea pigs are shown in FIG. 2. The test results show that the imidacloprid drops prepared in the examples are non-irritating to the intact and broken skin of rabbits, and there is no skin sensitization reaction to guinea pigs.

(4) Clinical Efficacy Test

The external preparation of imidacloprid prepared in the above example was commissioned to Huazhong Agricultural University to carry out the clinical efficacy test on dogs and cats naturally infected with lice and fleas in accordance with the “Quality Management Regulations for Non-Clinical Research of Veterinary Drugs” (GCP) of the Ministry of Agriculture and Rural Affairs of China.

(1) The clinical efficacy test on cats naturally infected with fleas is as follows:

124 cats (weight of less than 4 kg) infected with fleas and free of other diseases were collected. The selected animals were randomly divided into a test group and a control group. The test group was administrated with the external preparation of imidacloprid in Example 1 with the dosage of 0.4 mL. Once administered, the effective effect for fleas can be maintained for four weeks. The control group was administrated with Bayer's Wangdijing® imidacloprid drops, the dosage was 0.4 mL, once used, the effective effect for fleas could be maintained for four weeks. The number of fleas in the test group and the control group was counted on Day 0, 2, 7, 14, and 28 respectively. The results obtained are shown in Table 4.

TABLE 4
Clinical efficacy test on cats naturally infected with fleas

| | cure rate of control group | | cure rate of test group | |
|---|---|---|---|---|
| | (n = 62) | | (n = 62) | |
| | (number of cured animals/ | | (number of cured animals/ | |
| | number of infected animals) | | number of infected animals) | |
| Day 0 | 0% | (0/62) | 0% | (0/62) |
| Day 2 | 97% | (60/62)^{##} | 97% | (60/62) ** |
| Day 7 | 100% | (60/62)^{##} | 100% | (60/62) ** |
| Day 14 | 100% | (60/62)^{##} | 100% | (60/62) ** |
| Day 28 | 100% | (60/62)^{##} | 100% | (60/62) ** |

Note:
^{##}means that the number of test days is compared with that of Day 0, and the difference is very significant, P ≤ 0.01;
** means that the number of test days is compared with that of Day 0, and the difference is very significant, P ≤ 0.01.

(2) The clinical efficacy test on dogs naturally infected with lice is as follows:

135 dogs infected with lice and free of other diseases were collected. The administered dosage was: <4 kg, 0.4 ml; >4 and <10 kg, 1.0 ml; >10 and <25 kg, 2.5 ml; >25 and <40 kg, 4.0 ml.

The selected animals were randomly divided into a test group and a control group. The test group was administrated with the external preparation of imidacloprid in Example 1 according to the administered dosage. Once administered, the effective effect for lice can be maintained for four weeks. The control group was administrated with Bayer's Wangdijing® imidacloprid drops according to the administered dosage, once used, the effective effect for lice could be maintained for four weeks. The number of lice in the test group and the control group was counted on Day 0, 2, 7, 14, and 28 respectively. The results are shown in Table 5.

TABLE 5
Clinical efficacy test on dogs naturally infected with lice

| | cure rate of control group | | cure rate of test group | |
|---|---|---|---|---|
| | (n = 67) | | (n = 68) | |
| | (number of cured animals/ | | (number of cured animals/ | |
| | number of infected animals) | | number of infected animals) | |
| Day 0 | 0% | (0/67) | 0% | (0/68) |
| Day 2 | 97% | (65/67)^{##} | 94% | (64/68) ** |
| Day 7 | 100% | (67/67)^{##} | 100% | (68/68) ** |
| Day 14 | 100% | (67/67)^{##} | 100% | (68/68) ** |
| Day 28 | 100% | (67/67)^{##} | 100% | (68/68) ** |

Note:
^{##}means that the number of test days is compared with that of Day 0, and the difference is very significant, P ≤ 0.01;
** means that the number of test days is compared with that of Day 0, and the difference is very significant, P ≤ 0.01.

(3) The clinical efficacy test on dogs naturally infected with fleas is as follows:

128 dogs infected with fleas and free of other diseases were collected. The administered dosage was: <4 kg, 0.4 ml; >4 and <10 kg, 1.0 ml; >10 and <25 kg, 2.5 ml; >25 and <40 kg, 4.0 ml.

The selected animals were randomly divided into a test group and a control group. The test group was administrated with the external preparation of imidacloprid in Example 1 according to the administered dosage. Once administered, the effective effect for fleas can be maintained for four weeks. The control group was administrated with Bayer's Wangdijing® imidacloprid drops according to the administered dosage, once used, the effective effect for fleas could be maintained for four weeks. The number of fleas in the test group and the control group was counted on Day 0, 2, 7, 14, and 28 respectively. The results are shown in Table 6.

TABLE 6
Clinical efficacy test on dogs naturally infected with fleas

| | cure rate of control group | | cure rate of test group | |
|---|---|---|---|---|
| | (n = 64) | | (n = 64) | |
| | (number of cured animals/ | | (number of cured animals/ | |
| | number of infected animals) | | number of infected animals) | |
| Day 0 | 0% | (0/64) | 0% | (0/64) |
| Day 2 | 97% | (62/64)^{##} | 95% | (61/64) ** |
| Day 7 | 100% | (64/64)^{##} | 100% | (64/64) ** |
| Day 14 | 100% | (64/64)^{##} | 100% | (64/64) ** |
| Day 28 | 100% | (64/64)^{##} | 100% | (64/64) ** |

Note:
^{##}means that the number of test days is compared with that of Day 0, and the difference is very significant, P ≤ 0.01;
** means that the number of test days is compared with that of Day 0, and the difference is very significant, P ≤ 0.01.

The results of clinical tests show that the cure rate of external preparation of imidacloprid against natural infections of lice and fleas in dogs and cats reaches more than 90%, and no adverse reactions are found during use, indicating that the external preparation of imidacloprid prepared by the present disclosure has better safety and effectiveness in pet clinics.

### COMPARATIVE EXAMPLE

The antioxidant in Example 1 was replaced with equivalent amount of polyphenols, vitamin C, vitamin E, citric acid, sodium bisulfite, and the stability of the external preparation of imidacloprid obtained under high temperature and light irradiation was tested, and the test time was 10 days. The results are shown in Table 7.

TABLE 7
Stability test results of Comparative Example

content change compared with 0 day (%)

| | high temperature | light irradiation |
|---|---|---|
| antioxidant | (60° C.) | (45001x ± 5001x) |
| Example 1 | −0.4% | −0.2% |
| polyphenols | −1.5% | −0.5% |
| vitamin C | −5.8% | −2.1% |
| vitamin E | −2.5% | −1.1% |
| citric acid | −15.8% | −14.6% |
| sodium bisulfite | −6.5% | −2.5% |

It can be seen from the above test that the external preparation of imidacloprid provided by the present disclosure has good stability.

The above are only the preferred embodiments of the present disclosure. It should be pointed out that for the persons skilled in the art, without departing from the principle of the present disclosure, several improvements and modifications can be made, and these improvements and modifications should also be regarded as the protection scope of the present disclosure.

1. An external preparation of imidacloprid, including following components in percentage by mass:
Imidacloprid technical 5-15%;
Antioxidant 0.05-0.2%;
Alcohol solvent 80-90%;
Stabilizer 4.95-10%;
wherein antioxidant is one or more of dibutyl hydroxytoluene, hydroxymethyl, propyl hydroxybenzoate and butylated hydroxyanisole; and wherein
the stabilizer is one or more of propylene carbonate, povidone and copovidone.

2. The external preparation of imidacloprid according to claim 1,
wherein including following components in percentage by mass:
Imidacloprid technical 8-12%;
Antioxidant 0.1-0.15%;
Alcohol solvent 82-88%;
Stabilizer 6-8%.

3. The external preparation of imidacloprid according to claim 1, wherein the alcohol solvent is one or more of benzyl alcohol, propylene glycol and isopropanol.

4. The external preparation of imidacloprid according to claim 2, wherein the alcohol solvent is one or more of benzyl alcohol, propylene glycol and isopropanol.

5. The external preparation of imidacloprid according to claim 1, wherein a dosage form of the external preparation of imidacloprid is a drop.

6. The external preparation of imidacloprid according to claim 2, wherein the dosage form of the external preparation of imidacloprid is a drop.

7. A method for preparing the external preparation of imidacloprid according to claim 1, comprising steps of:
Mixing the imidacloprid technical, antioxidant, alcohol solvent and stabilizer to obtain the external preparation of imidacloprid.

8. The preparation method according to claim 7, wherein including following components in percentage by mass:
Imidacloprid technical 8-12%;
Antioxidant 0.1-0.15%;
Alcohol solvent 82-88%;
Stabilizer 6-8%.

9. The preparation method according to claim 7, wherein the alcohol solvent is one or more of benzyl alcohol, propylene glycol and isopropanol.

10. The preparation method according to claim 8, wherein the alcohol solvent is one or more of benzyl alcohol, propylene glycol and isopropanol.

11. The preparation method according to claim 7, wherein a dosage form of the external preparation of imidacloprid is a drop.

12. The preparation method according to claim 8, wherein a dosage form of the external preparation of imidacloprid is a drop.

13. The preparation method according to claim 7, wherein, the mixing is conducted by:
Heating and mixing the alcohol solvent, antioxidant and imidacloprid technical to obtain a mixed solution and;
Adding the stabilizer to the mixed solution to obtain the external preparation of imidacloprid.

14. The preparation method according to claim 13, wherein a temperature of the heating and mixing is 35-40° C.

15. Use of the external preparation of imidacloprid according to claim 1.

16. The use according to claim 15, wherein including following components in percentage by mass:
Imidacloprid technical 8-12%;
Antioxidant 0.1-0.15%;
Alcohol solvent 82-88%;
Stabilizer 6-8%.

17. The use according to claim 15, wherein the alcohol solvent is one or more of benzyl alcohol, propylene glycol and isopropanol.

18. The use according to claim 15, wherein a dosage form of the external preparation of imidacloprid is a drop.

19. The use according to claim 15, wherein the ectoparasites are one or more of lice, fleas and ticks.

20. The use according to claim 15, wherein the external preparation of imidacloprid is used for pets.
